(19) United States
(12) Patent Application Publication
(10) Pub. No.: US 2003/0195242 A1
(43) Pub. Date: Oct. 16, 2003

(54) Use of Cox-2 inhibitors to prevent recurrences of herpesvirus infections

(76) Inventors: Herbert E. Kaufman, New Orleans, LA (US); Bryan M. Gebhardt, New Orleans, LA (US); Emily D. Varnell, New Orleans, LA (US)

Correspondence Address: PATENT DEPARTMENT, TAYLOR, PORTER, BROOKS & PHILLIPS, L.L.P, P.O. BOX 2471, BATON ROUGE, LA, 70821-2471, US

(21) Appl. No.: 10/122,666

(22) Filed: Apr. 15, 2002

(51) Int. Cl. A61K 31/415; A61K 31/365

(52) U.S. Cl. 514/406; 514/473

(57) ABSTRACT

Selective inhibitors for COX-2 were discovered to prevent the reactivation of viruses that cause latent infections such as herpes simplex virus (HSV-1 and HSV-2). Using mice with a latent infection of HSV, which is subject to reactivation when heat-stressed, a selective COX-2 inhibitor (celecoxib) was shown to significantly suppress viral reactivation in the eye when the inhibitor was administered either by intraperitoneal injection or orally. Acetylsalicylic acid, a nonspecific cyclooxygenase inhibitor, was also found to suppress viral reactivation in this heat-stress mouse model. The COX-2 specific inhibitor, celecoxib, was more effective in preventing viral recurrence than was the nonspecific cyclooxygenase inhibitor aspirin. The use of selective inhibitors of COX-2 to inhibit the recurrence of latent viral infections will be more effective and have fewer side effects than the nonspecific inhibitors. In addition, selective inhibitors of COX-2 can be combined with other known antiviral compounds.

The development of this invention was partially funded by the Government under U.S. Public Health Service grants EY02672 and EY02377 (departmental core grant) from the National Eye Institute, National Institutes of Health. The Government has certain rights in this invention.

This invention pertains to a method to prevent recurrent infections by herpesviruses, e.g., herpes simplex viruses types 1 and 2, by administering an effective amount of a inhibitor known to be specific for inhibiting the enzyme cyclooxygenase-2 (COX-2).

Recurrences due to a latent infection by a herpesvirus are common. The herpesviruses that can cause latent infections include herpes simplex viruses types 1 and 2 (HSV-1 and 2), cytomegalovirus, Epstein Barr virus, and varicella zoster virus. Recurrences due to HSV-1 and 2 are debilitating diseases. Recurrent genital herpes virus infection is painful, transmissible to sexual partners, and emotionally debilitating. Recurrent ocular herpetic infection is also painful, potentially transmissible, and may lead to blindness. HSV infections are common throughout the world both in industrialized and underdeveloped countries. To date, vaccines to prevent initial viral infection and prevent recurrent viral infection have not proven to be effective. There is a need for safe and effective prophylactic drugs to prevent recurrent infections of HSV and other herpesviruses.

Some of the antivirals in current use, such as acyclovir, famcylovir, and others, have been tested for their capacity to prevent recurrent viral infection in high risk patients. The data resulting from such studies indicate that prophylactic antiviral therapy can reduce but does not eliminate viral recurrences. See Herpetic Eye Disease Study Group, “Oral acyclovir for herpes simplex virus eye disease: Effect on prevention of epithelial keratitis and stromal keratitis,” Arch. Ophthalmol., vol. 118, pp. 1030-1036 (2000). However, chronic antiviral therapy is not only expensive, but also may have toxic side effects. Other viral inhibitors are known that act as enzyme inhibitors, e.g., viral thymidine kinase inhibitors.

The role of prostaglandin in mediating herpes simplex virus type 1 (HSV-1) reactivation has been investigated because many factors found to precipitate a recurrence of an HSV infection (i.e., exposure to ultraviolet light, trauma, and fever) are associated with an increase in prostaglandin production. See K. F. Trofatter et al., “Effect of prostaglandin and cyclic adenosine 3′,5′-monophosphate modulators on herpes simplex virus growth and interferon response in human cells,” Infect. Immun., vol. 27, pp. 158-167 (1980). In in vitro experiments, prostaglandins were found to enhance the spread of HSV-1 in cell culture and to induce viral reactivation. See W. A. Blyth et al., “Reactivation of herpes simplex virus infection by ultraviolet light and possible involvement of prostaglandin,” J. gen. Virol., vol. 33, pp. 547-550 (1976); T. J. Hill et al., “An alternative theory of herpes-simplex recurrence and a possible role for prostaglandin,” The Lancet, vol. Feb 21, pp. 397-399 (1976); and D. A. Harbour et al., “Prostaglandin enhance spread of herpes simplex virus in cell cultures,” J. gen. Virol., vol. 41, pp. 87-95 (1978). Moreover, the addition of prostaglandin inhibitors to cell cultures has been reported to significantly reduce viral replication. See A. A. Newton, “Effect of cyclic nucleotides on the response of cells to infection by various herpesviruses,” In: Oncogenesis and Herpes Viruses III (G deThe, W Henle, and F Rapp, eds.), I.A.R.C., Lyon, pp 381-387 (1978); A. A. Newton, “Inhibitors of prostaglandin synthesis as inhibitors of herpes simplex virus replication,” Adv. Ophthal., vol. 38, pp. 58-63 (1979); and A. A. Newton, “Prostaglandin and the replication of herpes simplex type 1,” In: Herpesvirus. Clinical, Pharmacological and Basic Aspects, (H Shiota, Y-C Cheng, W H Prusoff, eds.),. Excerpta Medica, Amsterdam-Oxford-Princeton, pp 192-200. (1982).

In later studies, the topical use of latanoprost, a prostaglandin F₂ alpha analog, was shown to stimulate the recurrence of herpes simplex keratitis both in humans and rabbits. See M. Wand et al., “Latanoprost and herpes simplex keratitis,” Am. J. Ophthalmol., vol. 127, pp. 602-604 (1999); and H. E. Kaufman et al., “Latanoprost increases the severity and recurrence of herpetic keratitis in the rabbit,” Am. J. Ophthalmol., vol. 127, pp. 531-536 (1999).

However, the effect of prostaglandins may depend on the specific prostaglandin investigated. For example, prostaglandin alpha 2 has been shown to inhibit in vitro HSV-1 replication in human and rabbit cells. See W. J. O'Brien et al., “Assessment of antiviral activity, efficacy, and toxicity of prostaglandin A₂ in a rabbit model of herpetic keratitis, ” Antimicrob. Agents Chemother., vol. 40, pp. 2327-2331 (1996). However, in rabbit in vivo experiments, prostaglandin alpha 2 was not found to be an effective inhibitor of viral activity, and in fact caused a more severe acute viral infection of the cornea.

The mechanisms of stress-induced viral reactivation are little known. See W. P. Halford, “Mechanisms of Herpes Simplex Virus Type 1 Reactivation,” J. Virol., vol. 70, pp. 5051-5060 (1996). The role of prostaglandins in stimulating viral reactivation and promoting viral replication have not been completely elucidated. One report examined the action of prostaglandins in enhancing replication of HSV-1 by adding phosphodiesterase inhibitors, which were found to delay the growth of virus, inhibit cell-to-cell spread, and suppress the production of interferon by human mononuclear leukocytes. Because the addition of various prostaglandins had no direct stimulatory effect on HSV replication, the suggestion was made that prostaglandins may exacerbate HSV infection indirectly by inhibiting both the action and the production of interferon. See K. F. Trofatter et al., 1980.

Earlier observations had indicated that certain nonsteroidal anti-inflammatory drugs, mefenamic and indomethacin, inhibited virally-induced inflammation in herpetic epithelial lesions, inhibited a local spread of lesions, and shorted the duration of the disease process. See A. D. Inglot et al., “Topical treatment of cutaneous herpes simplex in humans with the non-steroid antiinflammatory drugs: mefenamic acid and indomethacin in dimethylsulfoxide,” Archivum. Immunologiae et Therapic Experimentalis, vol. 19, pp. 555-566 (1971); and Harbour et al., 1978. In other experiments, the trigeminal ganglia of mice were incubated in vitro in various concentrations of indomethacin, tetracaine, mepacrine, and mefenamic acid, which were found to inhibit the reactivation of virus. See I. Kurane et al., “Inhibition by indomethacin of in vitro reactivation of latent herpes simplex virus type 1 in murine trigeminal ganglia,” J. gen. Virol., vol. 65, pp. 1665-1674 (1984).

Other anti-inflammatory drugs have been proposed to act as antiviral agents, e.g., acetylsalicylic acid. See V. Primache et al., “In vitro activity of acetylsalicylic acid on replication of varicella-zoster virus,” Microbiologica, vol. 21, pp. 397-401 (1998); and I. Karadi et al., “Aspirin in the management of recurrent herpes simplex virus infection,” Ann. Int. Med., vol. 128, pp. 696-697 (1998). In addition, the nonspecific cyclooxygenase inhibitors ibuprofen and indomethacin were reported to reduce the incidence and frequency of recurrent herpes simplex virus infection. See M. Wachsman et al., “The prophylactic use of cyclooxygenase inhibitors in recurrent herpes simplex infections,” Br. J. Dermatol., vol. 123, pp. 375-380 (1990); and Newton, 1979.

One of the pathways in the synthesis of prostaglandin is the cyclooxygenase pathway. Cyclooxygenase (COX) is the rate-limiting enzyme in the production of prostaglandin from arachidonic acid. Cyclooxygenase is known to exist in two forms, cyclooxygenase-1 (COX-1) and cyclooxygenase-2 (COX-2). COX-1 is a constitutively-expressed enzyme, which is expressed in a majority of tissues, while COX-2 is constitutively expressed only in a few tissues, e.g., brain tissue. COX-2 has been shown to be induced by mediators produced due to inflammation, e.g., cytokines, and by chronic exposure to ultraviolet radiation. See U.S. Pat. No. 6,048,850.; and M. Athar et al., “Ultraviolet B (UVB)-induced COX-2 expression in murine skin: an immunohistochemical study,” Biochem. Biophys. Res. Comm., vol. 280, pp. 1042-1047 (2001). Nonspecific cyclooxygenase inhibitors that inhibit both COX-1 and COX-2 are known to cause many side effects because the inhibition of COX-1 results in a decrease in prostaglandins, which are necessary for normal body function. On the other hand, cyclooxygenase inhibitors specific for COX-2 have fewer side effects on normal body functions. See U.S. Pat. No. 6,048,850.

In recent years, several compounds have been identified that are selective inhibitors of COX-2, e.g., rofecoxib (also known as “refecoxib”); etoricoxib (also known as “MK-663”); NS-398; DuP-697; SC-58125; DFU; L-745,337; RS 57067; celecoxib (also known as “SC-58635”); valdecoxib; meloxicam; flosulide; nimesulide; and parecoxib. See U.S. Pat. Nos. 6,180,651; 6,004,994; 5,972,950; 5,859,036; 5,866,596; and 5,686,460; International Application Nos. WO 01/52897 A2; WO 01/28548 A1; and WO 01/15687 A1.

We have discovered that inhibitors that are specific for COX-2 inhibit the recurrence of a latent herpesvirus infection, such as herpes simplex virus (HSV-1 and HSV-2). Using mice with a latent infection of HSV, which is subject to reactivation when heat-stressed, a selective COX-2 inhibitor (celecoxib) was shown to significantly suppress viral reactivation in the eye when the inhibitor was administered either by intraperitoneal injection or orally. Acetylsalicylic acid, a nonspecific cyclooxygenase inhibitor, was also found to suppress viral reactivation in this heat-stress mouse model. The COX-2 specific inhibitor, celecoxib, was more effective in preventing viral recurrence than was the nonspecific cyclooxygenase inhibitor aspirin. The use of selective inhibitors of COX-2 to inhibit the recurrence of herpesvirus infections will be more effective and have fewer side effects than the nonspecific inhibitors. In addition, selective inhibitors of COX-2 can be combined with other known antiviral compounds.

As used herein, the term “selective COX-2 inhibitor” refers to a compound, at an appropriate dosage, that selectively inhibits COX-2, and does not significantly inhibit COX-1. Examples of currently known COX-2 inhibitors include rofecoxib (also known as “refecoxib”); etoricoxib (MK-663); NS-398; DuP-697; SC-58125; DFU; L-745,337; RS 57067; celecoxib (SC-58635); valdecoxib; meloxicam; flosulide; nimesulide; and parecoxib.

The selective COX-2 inhibitor may be administered to a patient by any suitable means, including parenteral, subcutaneous, intrapulmonary, intranasal, topical and oral administration. Parenteral infusions include intramuscular, intravenous, intraarterial, or intraperitoneal administration. The selective COX-2 inhibitor may also be administered transdermally, for example, in the form of a slow-release subcutaneous implant, or orally in the form of capsules, powders, or granules. It may also be administered by inhalation. The selective COX-2 inhibitor may be particularly administered topically since other drugs are known to be active topically, e.g., latanoprost. See Kaufman et al., 1999.

Pharmaceutically acceptable carrier preparations for parenteral administration include sterile, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. The active therapeutic ingredient maybe mixed with excipients that are pharmaceutically acceptable and are compatible with the active ingredient. Suitable excipients include water, saline, dextrose, glycerol and ethanol, or combinations thereof. Intravenous vehicles include fluid and nutrient replenishers, electrolyte replenishers, such as those based on Ringer's dextrose, and the like. Preservatives and other additives may also be present such as, for example, antimicrobials, anti-oxidants, chelating agents, inert gases, and the like.

The form may vary depending upon the route of administration. For example, compositions for injection may be provided in the form of an ampule, each containing a unit dose amount, or in the form of a container containing multiple doses.

The selective COX-2 inhibitor may be formulated into therapeutic compositions as pharmaceutically acceptable salts. These salts include the acid addition salts formed with inorganic acids such as, for example, hydrochloric or phosphoric acid, or organic acids such as acetic, oxalic, or tartaric acid, and the like. Salts also include those formed from inorganic bases such as, for example, sodium, potassium, ammonium, calcium or ferric hydroxides, and organic bases such as isopropylamine, trimethylamine, histidine, procaine and the like.

Controlled delivery may be achieved by admixing the active ingredient with appropriate macromolecules, for example, polyesters, polyamino acids, polyvinyl pyrrolidone, ethylenevinylacetate, methylcellulose, carboxymethylcellulose, prolamine sulfate, or lactide/glycolide copolymers. The rate of release of the selective COX-2 inhibitor maybe controlled by altering the concentration of the macromolecule.

Another method for controlling the duration of action comprises incorporating the selective COX-2 inhibitor into particles of a polymeric substance such as a polyester, peptide, hydrogel, polylactide/glycolide copolymer, or ethylenevinylacetate copolymers. Alternatively, the selective COX-2 inhibitor may be encapsulated in microcapsules prepared, for example, by coacervation techniques or by interfacial polymerization, for example, by the use of hydroxymethylcellulose or gelatin-microcapsules or poly(methylmethacrylate) microcapsules, respectively, or in a colloid drug delivery system. Colloidal dispersion systems include macromolecule complexes, nanocapsules, microspheres, beads, and lipid-based systems including oil-in-water emulsions, micelles, mixed micelles, and liposomes.

The term “therapeutically effective amount” as used herein refers to an amount of the selective COX-2 inhibitor sufficient to decrease the frequency of reactivation of a herpesvirus. The term “therapeutically effective amount” therefore includes, for example, an amount of a selective COX-2 inhibitor sufficient to reduce the recurrence of HSV, and preferably to reduce by at least 50%, and more preferably to reduce by at least 90%, the frequency of HSV recurrence. The dosage ranges for the administration of the selective COX-2 inhibitor are those that produce the desired effect. A person of ordinary skill in the art, given the teachings of the present specification, may readily determine suitable dosage ranges. The dosage can be adjusted by the individual physician in the event of any contraindications. In any event, the effectiveness of treatment can be determined by monitoring the recurrence of the herpesvirus by methods well known to those in the field, and as described below.

The selective COX-2 inhibitor can also be combined with other compounds known to be effective in decreasing the recurrence of herpesviruses, e.g., acyclovir, famcyclovir, viral thymidine kinase inhibitor, and other partially effective HSV recurrence inhibitors.

### EXAMPLE 1

Materials and Methods

Virus and Virological Analyses

The McKrae strain of HSV-1 was propagated in Vero cells (ATCC No. CCL-81, American Type Culture Collection, Manassass, Va.), titered by viral plaque assay, and stored at −70° C. until used to infect animals.

Ocular swabs were immersed in 0.2 ml of complete tissue culture medium consisting of RPMI-1640, 10% fetal bovine serum (FBS), and an antibiotic-antimycotic mixture (Life Technologies, Grand Island, N.Y.). The swabs in culture medium were stored at 4° C. until testing by plaque assay. The determination of infectious virus in the cornea and in the trigeminal ganglia of animals were performed in a similar manner. Pairs of corneas and ganglia from individual mice were placed in separate tubes in 0.2 ml of complete medium, homogenized, clarified by centrifugation at 14,000×g for 5 minutes and the supernatant tested for infectious virus on Vero cells.

Animals and Animal Infection

Male BALB/c strain mice (The Jackson Laboratory, Bar Harbor, Me.) were infected at 5 weeks of age by topical application of 5×10⁵ plaque forming units of the McKrae strain HSV-1 following superficial scratching of the cornea. Documentation of the success of viral infection was determined by culturing ocular swabs taken on days 3 and 5 after infection. Animals without infectious virus on both days 3 and 5 in both eyes were excluded from use in these experiments. Thirty days after infection the eyes were swabbed to establish that infectious virus was no longer present on the ocular surface. Groups of uninfected age and sex matched mice were used as control animals in this study.

Quantitation of Viral DNA

Quantitation of viral DNA in tissue homogenates was made using the polymerase chain reaction (PCR), as previously reported. See B. M. Gebhardt et al., “9-(4-hydroxybutyl)-N²-phenylguanine (HBPG), a thymidine kinase inhibitor, suppresses herpes virus reactivation in mice,” Antiviral Res., vol. 30, pp. 87-94 (1996); and J. M. Hill et al., “Quantitative analysis of polymerase chain reaction products by dot blot,” Analyt. Biochem., vol. 235, pp. 44-48 (1996). In brief, the procedure involves comparing DNA extracted from experimental and control tissue samples containing viral DNA, and performing serial dilutions with DNA from homologous tissue homogenates of uninfected animals. Using a log dilution series and previously determined quantities of viral DNA, it is possible to obtain a series of curves which permit the sensitive and reproducible determination of viral DNA in experimental samples. The sensitivity of this procedure permits the determination of differences of 100 viral genome equivalents per sample. In each experimental series, standard curves were conducted along with the analyses of the tissue homogenates from experimental (drug treated) and control (placebo treated) mice.

Statistical Methods

Analysis of normally distributed data was performed using a one-way ANOVA and Tukey's posthoc T-test. The correlation coefficient and its statistical significance were determined by regression analysis. The statistical significance of HSV-1 reactivation in mice was evaluated by the COX-Mantel test. In these statistical analyses P≦0.05 was considered the minimum significant difference to reject H₀:x₁=x₂. All experiments were repeated a minimum of 3 times in order to assess the reproducibility of the results.

### EXAMPLE 2

Effect of Acetylsalicylic Acid (ASA) on Heat-Stress Reactivation of HSV

To test the effect of acetylsalicylic acid (ASA) on reactivation of HSV induced by heat stress, two sets of experiments were performed. In one set, groups of mice with latent HSV were immersed in 43° C. water up to their necks for 10 minutes. Following the hyperthermic stress, the animals were dried and given an intraperitoneal (IP) injection of 0.1 mg of ASA (Sigma Chemical Co., St. Louis, Mo.) suspended in 0.1 ml saline. At 8 and 16 hours after hyperthermia, the animals were again treated with ASA. Control mice were given IP injections of saline on the same schedule. At 24 hr after hyperthermic stress, the eyes were swabbed, the mice were sacrificed, and the corneas and trigeminal ganglia analyzed for infectious virus and viral DNA.

In a second experiment, groups of mice were treated IP for 3 days prior to hyperthermic stress at 8 hr intervals on each of the 3 days. The animals were then heat stressed, treated immediately after stress with ASA, and treated at 8 and 16 hr after hyperthermic stress. Control mice were given IP injections of saline on the same schedule. Two additional groups of mice were prophylactically treated by oral administration of 0.1 mg ASA or saline on the same dose schedule. In each experiment, animals were subdivided into those which were hyperthermically stressed and those which were not stressed. As above, the eyes of these animals were swabbed at 24 hr after stress and then sacrificed for analysis of infectious virus and viral DNA in their corneas and trigeminal ganglia.

Control mice for each of these experiments included animals which were latent for HSV-1 but which were not heat stressed. Subsets of this control group included animals placebo-treated and animals treated with ASA. Two additional control groups of animals were those which were not latent for HSV-1, but which were either treated with placebo or ASA on the schedules outlined above.

In each experiment the body temperature of ASA-treated and saline-treated mice was determined before and after heat stress with a microprobe thermometer and rectal probe (World Precision Instruments, Sarasota, Fla.). The mean body temperature for ASA-treated mice before heat stress was 37° C.±0.2 and immediately after heating was 40° C.±0.3. The mean body temperature of saline-treated mice before heat stress was 37.5° C.+0.3 and immediately after heating was 40.2° C.±0.2. Thus, there was no significant effect of ASA prophylaxis on body temperature; both saline-treated and drug-treated mice experienced similar elevations in body temperature following heat stress.

Treatment of mice with ASA beginning at the time of hyperthermic stress had a significant effect in lowering the number of animals with infectious virus on their ocular surface 24 hours after reactivation (Table 1). Compared to heat-stressed animals treated with saline, fewer of the stressed ASA-treated animals had infectious virus on their ocular surface. Three different sets of controls did not have infectious virus on their ocular surface: control mice that were heat-stressed but not infected, control mice that were neither infected nor heat-stressed, and latent mice that were infected but not heat stressed.

TABLE 1
Effect of ASA Treatment on the Numbers of Ocular Surface Swabs
that were Positive for Herpes Simplex Virus*

| Treatment | Infectious Virus (no. positive/total) | | | | | |
|---|---|---|---|---|---|---|
| Groups | Experiment 1 | | Experiment 2 | | Experiment 3 | |
| Heat Stressed‡ | | | | | | |
| +ASA | 2/12 | (17%) | 3/12 | (25%) | 3/12 | (25%) |
| −ASA | 8/12 | (67%) | 9/12 | (75%) | 7/12 | (58%) |
| Not Stressed | | | | | | |
| +ASA | 0/10 | (0%) | 0/8 | (0%) | 0/10 | (0%) |
| −ASA | 0/12 | (0%) | 0/12 | (0%) | 0/8 | (0%) |

*Ocular surface swabs were obtained 24 hrs after heat stress and at the same time from unstressed controls. The numbers are the swabs which contained virus over the total number of swabs. The numbers in parentheses are the percent of cultures which contained virus.
‡There were statistically significantly fewer virus-containing cultures in the acetylsalicylic acid (ASA)-treated groups compared to the saline-treated groups, p < 0.05.

The number of homogenates of corneal tissue and trigeminal ganglia from ASA-treated animals that contained infectious virus was low, but statistically significantly from the number of homogenates from stressed animals treated with saline (Table 2).

TABLE 2
Effect of ASA Treatment on Virus in the Cornea
and Trigeminal Ganglion*

| Treatment Groups | Infectious Virus (no. positive/total) | |
|---|---|---|
| Cornea | | |
| +ASA | 5/36 | (14%) |
| −ASA | 11/36 | (31%) |
| Trigeminal Ganglia | | |
| +ASA | 8/36 | (22%) |
| −ASA | 15/36 | (42%) |

*Twenty-four hours after hyperthermic stress mice were sacrificed and their corneas and ganglia homogenized and tested for infectious virus. The numbers represent the number of cultures which were positive for virus over the number of homogenates tested. The numbers in parentheses are the percent of cultures which contained virus. There were significantly fewer virus-containing cultures in the ASA-treated groups compared to the saline-treated groups, p < 0.05.

### EXAMPLE 3

Effect of Acetylsalicylic Acid Prophylaxis on Viral Reactivation

To test for a prophylactic effect of ASA, mice were treated either IP or orally with ASA for three days prior to receiving the reactivation hyperthermic stimulus. These mice had significantly lower levels of infectious virus in their ocular tear film compared to placebo-treated mice which underwent hyperthermically induced reactivation. (Data not shown) Control animals that were latent for virus but not heat-stressed and animals that were heat-stressed but not infected did not have infectious virus in their ocular tear film (Data not shown).

Homogenates of the corneas and trigeminal ganglia of mice that had been prophylactically treated with ASA had fewer infectious units as compared to homogenates from placebo-treated animals that had been heat-stressed (Table 3). Control mice that were infected but not stressed and mice that were stressed but not infected did not have infectious virus in the corneal tissue or in their trigeminal ganglia.

TABLE 3
Effect of Prophylactic ASA on Virus in the Cornea and
Trigeminal Ganglion*

| Treatment Groups^{‡} | Infectious Virus (no. positive/total) |
|---|---|
| IP | |
| Cornea | |
| +ASA | 2/10 (20%) |
| −ASA | 5/12 (42%) |
| Trigeminal Ganglia | |
| +ASA | 3/10 (30%) |
| −ASA | 7/12 (58%) |
| Oral | |
| Cornea | |
| +ASA | 2/14 (14%) |
| −ASA | 7/14 (50%) |
| Trigeminal Ganglia | |
| +ASA | 4/14 (29%) |
| −ASA | 9/14 (64%) |

*Homogenates of corneas and trigeminal ganglia obtained 24 hrs after stress were cultured for infectious virus; the numbers represent the number of cultures positive over the total cultures. The numbers in parentheses are the percent of cultures which contained virus. Prophylactic ASA significantly reduced the number of homogenates which contained virus, p < 0.05.
^{‡}IP, intraperitoneal; oral gavage.

### EXAMPLE 4

Viral DNA Concentration in the Cornea and Trigeminal Ganglion

The levels of viral DNA found in the corneas of ASA-treated animals were not different from the amounts found in either placebo-treated animals or latent animals which were not heat-stressed (Table 4). However, significantly less viral DNA was found in the trigeminal ganglia of ASA-treated mice when compared to placebo-treated mice (Table 4).

TABLE 4
Effect of ASA on Viral DNA Concentration in the Cornea
and Trigeminal Ganglion*

| Treatment | Tissue | Viral DNA (genome equivalents) | |
|---|---|---|---|
| Heat Stressed | | | |
| +ASA | cornea | 0.9 × 10³ | ± 0.08 |
| −ASA | cornea | 0.8 × 10³ | ± 0.05 |
| +ASA | TG | 2.1 × 10⁴ | ± 0.1 |
| −ASA | TG | 3.5 × 10⁶ | ± 0.3 |
| Not Stressed | | | |
| +ASA | cornea | <1 × 10² | |
| −ASA | cornea | <1 × 10² | |
| +ASA | TG | 1.5 × 10² | ± 0.09 |
| −ASA | TG | 1.7 × 10² | ± 0.06 |

*Corneas and ganglia from six mice were pooled for quantitative analysis of viral DNA. There were significant differences in the viral DNA concentration in the ganglia of ASA− treated compared to untreated mice, p < 0.05.

Administration of ASA prior to heat stress also had no effect on the viral DNA concentration in the cornea (Table 5). However, there was significantly less viral DNA in the ganglia of the ASA-treated mice when compared to placebo-treated mice. (Table 5). Viral DNA was not found in control mice that were not latently infected regardless of whether or not they were heat-stressed.

TABLE 5
Effect of ASA Prophylaxis on Viral DNA Concentration in the
Cornea and Trigeminal Ganglion*

| Treatment | Tissue | Viral DNA (genome equivalents) | |
|---|---|---|---|
| Heat Stressed | | | |
| +ASA | cornea | 0.4 × 10⁴ | ± 0.05 |
| −ASA | cornea | 0.6 × 10⁴ | ± 0.06 |
| +ASA | TG | 4.9 × 10⁴ | ± 0.6 |
| −ASA | TG | 3.6 × 10⁶ | ± 0.7 |
| Not Stressed | | | |
| +ASA | cornea | <1 × 10² | |
| −ASA | cornea | <1 × 10² | |
| +ASA | TG | 2.3 × 10² | ± 0.04 |
| −ASA | TG | 3.1 × 10² | ± 0.07 |

*Corneas and ganglia from six mice were pooled for quantitative analysis of viral DNA. There were significant differences in the viral DNA concentration in the ganglia of ASA− treated, heat-stressed mice compared to placebo-treated, heat-stressed mice, p < 0.05.

### EXAMPLE 5

### Effect of Selective COX-2 Inhibitor on Viral Recurrence

To test the effect of a selective COX-2 inhibitor on recurrence of HSV, female BALB/c mice (The Jackson Laboratory, Bar Harbor, Me.) at 4 weeks of age were used. The strain of HSV and the method of propagation were as described in Example 1.

The mice were infected by the topical ocular route, and the establishment of a corneal infection was documented by slit lamp examination and ocular swabs on day 3 after infection. Only animals which were successfully infected were retained for use in these experiments. Thirty days after infection, groups of 15 latently infected and 15 uninfected mice were treated with the COX 2 inhibitor, celecoxib (G. D. Searle & Co., Skokie, Ill.). The dose of celecoxib that would inhibit viral reactivation was determined in a preliminary experiment in which groups of mice were injected intraperitoneally with 0.1, 0.5, 1.0, and 5.0 mg of celecoxib four times daily. The mice were treated one day before heat stress and the day of the heat stress. The most effective dose in preventing viral recurrence was found to be 0.5 mg of celecoxib four times daily. This dosage was used in all subsequent experiments.

Two days prior to hyperthermic stress, uninfected mice and latently infected mice were treated four times daily with celecoxib at a concentration of 0.5 mg per treatment. The celecoxib suspension was given intraperitoneally in some groups of mice and orally in others. This treatment was continued on the day that animals were heat stressed by immersion into 43° C. water for 10 minutes. Twenty-four hours after the hyperthermic stress, the eyes were swabbed to culture any infectious virus, and the mice killed to collect the trigeminal ganglia. Ocular swabs were incubated in culture medium which was then transferred to Vero cell monolayers in 96 well culture plates. Units of Infectious virus were recorded as units of cytopathic effect over a period of seven days. Units of cytopathic effect were the number of plaques or dead Vero cells caused by the virus, i.e., one viral particle causes one unit of cytopathic effect (a plaque.). Pairs of trigeminal ganglia from each mouse were homogenized in 0.5 ml of tissue culture medium, and the homogenate tested for infectious virus by adding to Vero cell monolayers. The units of cytopathic effect were recorded over a seven day incubation period.

Because the celecoxib was administered by two different routes, intraperitoneally and orally, differences between these treatment approaches were included in the statistical analysis. Initially, the mean number of animals exhibiting reactivation in each of the groups were compared by a one way analysis of variance. Subsequently, Tukey's posthoc analysis test was performed to further confirm statistical differences between the treatment and control groups.

As shown in Table 6, treatment of animals with celecoxib intraperitoneally or orally reduced the frequency with which infectious virus was present on the ocular surface. Compared to untreated animals that were heat-stressed, celecoxib treatment by either route was statistically significantly lower. Control, uninfected mice that were heat-stressed did not indicate any virus on their ocular surfaces.

TABLE 6
Inhibition of Viral Reactivation by Celecoxib*

Infectious Virus on the Ocular
Surface*

| Treatment Groups | IP | Oral |
|---|---|---|
| celecoxib, stressed | 5/14‡ | 5/12‡ |
| placebo, stressed | 9/15‡ | 9/14‡ |
| celecoxib, not stressed | 0/12 | 0/14 |
| placebo, not stressed | 0/14 | 0/12 |

*Mice were treated four times daily with 0.5 mg celecoxib or placebo intraperitoneally (IP) or orally for two days. On the second day, the mice were heat stressed by immersion up to their necks in 43° C. water for ten minutes. Control mice were not stressed. Twenty-four hours later the mice were tested for virus in their trigeminal ganglia.
‡Significantly fewer of the stressed mice treated with celecoxib had infectious virus in their trigeminal ganglia (p < 0.05).

Evidence that the COX 2 inhibitor, celecoxib, inhibits viral reactivation in the peripheral nervous system (the trigeminal ganglion) of mice is shown in Table 7. Mice treated either intraperitoneally or orally prior to undergoing hyperthermic stress had lower titers of infectious virus in the trigeminal ganglia as compared to mice that were infected and heat-stressed but not treated with the drug. Control animals did not have latent virus in their ganglia and, following hyperthermic stress, no infectious virus was found in the ganglia of these animals.

TABLE 7
Inhibition of Viral Reactivation by Celecoxib*

| | Infectious Virus in the Trigeminal Ganglia* | |
|---|---|---|
| Treatment Groups | IP | Oral |
| celecoxib, stressed | 5/12‡ | 4/12‡ |
| placebo, stressed | 10/13‡ | 10/14‡ |
| celecoxib, not stressed | 0/12 | 0/10 |
| placebo, not stressed | 0/12 | 0/10 |

*Mice were treated four times daily with 0.5 mg celecoxib or placebo intraperitoneally (IP) or orally for two days. On the second day, the mice were heat stressed by immersion up to their necks in 43° C. water for ten minutes. Control mice were not stressed. Twenty-four hours later the mice were tested for virus on their ocular surface.
‡Significantly fewer of the stressed mice treated with celecoxib had infectious virus on their ocular surface (p < 0.05).

Thus the selective COX-2 inhibitor celecoxib reduced the recurrence of latent HSV-1 infection in the eye. The drug was effective when administered both orally and intraperitoneally. It is believed that other selective COX-2 inhibitors would reduce the recurrence of latent herpesvirus infection.

The complete disclosures of all references cited in this specification are hereby incorporated by reference. Also incorporated by reference are the entire disclosures of each of the following, none of which is believed to be prior art to this application: B. M. Gebhardt, Emily D. Varnell, and Herbert E. Kaufman, “Inhibition of Herpesvirus Reactivation by Acetylsalicylic Acid,” to be submitted to Amer. J. Ophthal.; B. M. Gebhardt, Emily D. Varnell, and Herbert E. Kaufman, “Inhibition of Cyclooxygenase 2 Synthesis Prevents Herpesvirus Reactivation,” to be submitted to the Journal of Ocular Pharmacology and Therapeutics; and J. M. Hill et al., “Gene expression analyzed by microarrays in HSV-1 latent mouse trigeminal ganglion following heat stress,” Virus Genes, vol. 23, pp. 273-280 (2001). In the event of an otherwise irreconcilable conflict, however, the present specification shall control.

We claim:

1. A method of inhibiting the recurrence of a latent herpesviral infection in a mammal, said method comprising administering to the mammal a therapeutically effective amount of a selective COX-2 inhibitor.

2. A method of claim 1, wherein the latent herpesviral infection is an infection caused by a herpesvirus selected from a group consisting of herpes simplex viruses types 1 and 2 (HSV-1 and 2), cytomegalovirus, Epstein Barr virus, and varicella zoster virus.

3. A method of claim 1, wherein the latent herpesviral infection is an infection caused by herpes simplex virus type 1.

4. A method of claim 1, wherein the latent herpesviral infection is an infection caused by herpes simplex virus type 2.

5. A method of claim 1, wherein the latent herpesviral infection is an infection caused by cytomegalovirus.

6. A method of claim 1, wherein the latent herpesviral infection is an infection caused by Epstein Barr virus.

7. A method of claim 1, wherein the latent herpesviral infection is an infection caused by varicella zoster virus.

8. A method of claim 1, wherein the latent viral infection is a herpesviral infection in an eye of the mammal.

9. The method of claim 1, wherein the selective COX-2 inhibitor is selected from a group consisting of rofecoxib; etoricoxib; NS-398; DuP-697; SC-58125; DFU; L-745,337; RS 57067; celecoxib; valdecoxib; meloxicam; flosulide; nimesulide; and parecoxib.

10. The method of claim 1, wherein the selective COX-2 inhibitor comprises celecoxib.

11. The method of claim 1, wherein the selective COX-2 inhibitor comprises valdecoxib.

12. The method of claim 1, wherein the selective COX-2 inhibitor comprises refecoxib.

13. The method of claim 1, wherein the selective COX-2 inhibitor comprises meloxicam.

14. The method of claim 1, wherein the selective COX-2 inhibitor comprises flosulide.

15. The method of claim 1, wherein the selective COX-2 inhibitor comprises nimesulide.

16. The method of claim 1, wherein the selective COX-2 inhibitor comprises parecoxib.

17. The method of claim 1, wherein the mammal is a human.

18. The method of claim 1, wherein said administering of the selective COX-2 inhibitor is performed by injection, oral administration, or topical administration.

19. The method of claim 1, wherein said administering of the selective COX-2 inhibitor is performed by injection.

20. The method of claim 1, wherein said administering of the selective COX-2 inhibitor is performed by oral administration.

21. The method of claim 1, wherein said administering of the selective COX-2 inhibitor is performed by topical administration.

22. The method of claim 1, additionally comprising the step of administering to the mammal another compound that decreases the severity or partially inhibits reactivation of herpes simplex virus.

23. The method of claim 22, wherein the additional compound is selected from a group consisting of acyclovir, famcyclovir, and viral thymidine kinase inhibitors.

24. The method of claim 22, wherein the additional compound comprises acyclovir.

25. The method of claim 22, wherein the additional compound comprises famcyclovir.

26. The method of claim 22, wherein the additional compound comprises a viral thymidine kinase inhibitor.
